(19) United States
(12) Patent Application Publication
(10) Pub. No.: US 2026/0002186 A1
(43) Pub. Date: Jan. 1, 2026

(54) METHOD FOR ENDOGENOUSLY EXTRACTING MYCOBACTERIUM SMEGMATIS PROTEIN NANOCAGE

(71) Applicant: Nankai University, Tianjin (CN)

(72) Inventors: Hongri GONG, Tianjin (CN); Ziqing ZHAO, Tianjin (CN)

(73) Assignee: Nankai University, Tianjin (CN)

(21) Appl. No.: 18/826,473

(22) Filed: Sep. 6, 2024

(30) Foreign Application Priority Data
Jul. 1, 2024 (CN) 202410865668.6

(51) Int. Cl. C12P 21/02; C12N 15/74

(52) U.S. Cl. CPC C12P 21/02; C12N 15/74; C12N 2800/101

(57) ABSTRACT

A method for endogenously extracting a Mycobacterium smegmatis protein nanocage is provided. The method includes the following steps: introducing a recombinant plasmid containing a CFP29 gene and a 1×Flag affinity tag into a Mycobacterium smegmatis strain to obtain a recombinant Mycobacterium smegmatis strain; extracting a total protein solution of the recombinant Mycobacterium smegmatis strain, and subjecting the total protein solution of the recombinant Mycobacterium smegmatis strain to Flag tag affinity column chromatography purification to obtain a crude extract of the Mycobacterium smegmatis protein nanocage; and subjecting the crude extract of the Mycobacterium smegmatis protein nanocage to gel exclusion chromatography purification to obtain a pure product of the Mycobacterium smegmatis protein nanocage. The method is simple to operate and convenient to implement during extraction and purification, and an obtained Mycobacterium smegmatis background protein nanocage has a high yield, excellent purity, and stable properties.

### CROSS REFERENCE TO RELATED APPLICATION

This patent application claims the benefit and priority of Chinese Patent Application No. 2024108656686, filed with the China National Intellectual Property Administration on Jul. 1, 2024, the disclosure of which is incorporated by reference herein in its entirety as part of the present application.

### REFERENCE TO SEQUENCE LISTING

A computer readable XML file entitled “Sequence Listing”, that was created on Jul. 29, 2024, with a file size of 25,262 bytes, contains the sequence listing for this application, has been filed with this application, and is hereby incorporated by reference in its entirety.

### TECHNICAL FIELD

The present disclosure belongs to the technical field of protein extraction and purification, and particularly relates to a method for endogenously extracting a Mycobacterium smegmatis protein nanocage.

### BACKGROUND

Cell compartmentalization can overcome many metabolic difficulties and physiological challenges, and is essential for the efficient and normal functioning of cells. Bacterial encapsulin, a novel protein nanocage discovered in recent years that is smaller in size and more delicately assembled, includes a coat protein and a cargo protein. The coat protein has a diameter of 25 nm to 42 nm and can self-assemble into a regular icosahedral structure, and may encapsulate one or more cargo proteins inside. The cargo protein has a sequence of 20 to 30 amino acids at its terminal, called a target peptide (TP), which can mediate specific packaging of the cargo protein, thereby giving the encapsulin specific physiological functions, such as resisting oxidative stress damage. Therefore, it is of great significance for the further modification and application of protein nanocages to obtain the protein nanocages by in vitro extraction and purification.

Previous studies have shown that a shell of the protein nanocage derived from a soil bacterium Mycobacterium smegmatis exhibits a regular icosahedral structure assembled from 60 identical subunits, each of which is 29 kDa in size. Since this protein can be secreted into a bacterial medium, this protein is also named culture filtrate protein (CFP29). The in vitro extraction and purification of a protein nanocage from the non-pathogenic bacterium Mycobacterium smegmatis is of great significance for studying structure, function, and application of the protein nanocage.

In the present disclosure, the morphology and functional mechanism of a Mycobacterium smegmatis protein nanocage are extracted and identified for the first time. However, the protein nanocage obtained by natural extraction methods has poor yield and low purity, making it difficult to meet the demands of subsequent extensive research, and to further limit potential application values.

### SUMMARY

In view of this, an objective of the present disclosure is to provide a method for endogenously extracting a Mycobacterium smegmatis protein nanocage. In the present disclosure, the method has a low purification cost and can obtain the soil bacterium Mycobacterium smegmatis protein nanocage with high yield, excellent purity, and stable properties in a background strain.

The present disclosure provides a method for endogenously extracting a Mycobacterium smegmatis protein nanocage, including the following steps:
introducing a recombinant plasmid containing a (FP29 gene and a 1×Flag affinity tag into a Mycobacterium smegmatis strain to obtain a recombinant Mycobacterium smegmatis strain; extracting a total protein solution of the recombinant Mycobacterium smegmatis strain, and subjecting the total protein solution of the recombinant Mycobacterium smegmatis strain to Flag tag affinity column chromatography purification to obtain a crude extract of the Mycobacterium smegmatis protein nanocage; and subjecting the crude extract of the Mycobacterium smegmatis protein nanocage to gel exclusion chromatography purification to obtain a pure product of the Mycobacterium smegmatis protein nanocage.

A construction process of the recombinant plasmid includes: transferring the (FP29 gene, an upstream 806 bp nucleotide sequence of the (FP29 gene, a GGS-Linker, the 1×Flag affinity tag, a streptomycin selection marker, and a downstream 1,578 bp nucleotide sequence of the (FP29 gene into a vector plasmid.

The recombinant plasmid can be constructed by PCR and homologous recombination.

In the homologous recombination, an upstream 806 bp nucleotide sequence of the (FP29 gene and a downstream 1,578 bp nucleotide sequence of the (FP29 gene are used as homologous arms;
the upstream 806 bp nucleotide sequence of the CFP29 gene is shown in SEQ ID NO: 5, and the downstream 1,578 bp nucleotide sequence of the CFP29 gene is shown in SEQ ID NO: 6; and
the recombinant plasmid is pUC19-CFP29-Str-Flag.

The Flag tag affinity column chromatography purification is conducted on a chromatographic column filled with an Anti-Flag filler; and the gel exclusion chromatography purification is conducted on a chromatographic column of Superose 6 increase10/300 gel exclusion.

The Flag tag affinity column chromatography purification includes impurity elution and elution that are conducted in sequence; an impurity eluent for the impurity elution includes 3-(N-morpholino) propanesulfonic acid, NaCl, and a detergent; and a eluent for the elution includes the 3-(N-morpholino) propanesulfonic acid, the NaCl, and a 1×Flag peptide.

The detergent includes polyoxyethylene lauryl ether (POELE).

An equilibrium buffer for the gel exclusion chromatography purification includes 3-(N-morpholino) propanesulfonic acid and NaCl.

The 3-(N-morpholino) propanesulfonic acid has a molar concentration of 20 mmol/L to 25 mmol/L and the NaCl has a molar concentration of 100 mmol/L to 110 mmol/L in the equilibrium buffer.

A process of extracting the total protein solution of the recombinant Mycobacterium smegmatis strain includes: culturing the recombinant Mycobacterium smegmatis strain, collecting a resulting recombinant Mycobacterium smegmatis bacterial cell, resuspending the recombinant Mycobacterium smegmatis bacterial cell with a basic buffer, conducting high-pressure cell disruption, and collecting a supernatant obtained by centrifugation to obtain the total protein solution.

The recombinant strain is cultured at 36° C. to 38° C. with shaking at 200 rpm to 250 rpm for 4 d to 6 d.

A medium of the recombinant strain includes an LB broth.

The bacterial cell is resuspended in a dosage ratio of 1:(5-8) between the bacterial cell and the basic buffer to obtain a precipitated bacterial cell; and

The basic buffer reagent includes 3-(N-morpholino) propanesulfonic acid, sodium chloride, and phenylmethylsulfonyl fluoride (PMSF).

Preferably, the basic buffer includes 20 mmol/L of the 3-(N-morpholino) propanesulfonic acid, 100 mmol/L of the sodium chloride, and 1 mmol/L of the PMSF, and has a pH value of 7.4.

Compared with the prior art, the present disclosure has the following beneficial effects:

The present disclosure provides a method for endogenously extracting a Mycobacterium smegmatis protein nanocage. A 1×Flag affinity purification tag is inserted into a C-terminal on a gene of the Mycobacterium smegmatis protein nanocage by homologous recombination, and then Flag tag affinity column chromatography purification and gel exclusion chromatography purification are combined. The method has the advantages of simple operations, convenience, and a short experimental cycle, and has low requirements for experimental instruments and equipment, thereby greatly reducing time and financial costs. The Mycobacterium smegmatis background protein nanocage extracted by the method has high yield, excellent purity, and stable properties, and is of great significance for subsequent research on the structure and function of Mycobacterium smegmatis protein nanocages and advantages thereof in drug delivery.

### BRIEF DESCRIPTION OF THE DRAWINGS

FIG. 1 shows a map of the recombinant plasmid pUC19-CFP29-Str-Flag in Example 1;

FIGS. 2A-2B show purification results of the Mycobacterium smegmatis protein nanocage in Example 2; where FIG. 2A is a gel exclusion chromatography column diagram, and FIG. 2B is a 12% SDS-PAGE electrophoresis diagram;

FIG. 3 shows a negative staining electron microscopy result of the Mycobacterium smegmatis protein nanocage in Example 2;

FIG. 4 shows a structural diagram of the Mycobacterium smegmatis protein nanocage in Example 2; and

FIGS. 5A-5B show direct purification results of a wild-type Mycobacterium smegmatis protein nanocage in a comparative example.

### DETAILED DESCRIPTION OF THE EMBODIMENTS

The present disclosure provides a method for endogenously extracting a Mycobacterium smegmatis protein nanocage, including the following steps: introducing a recombinant plasmid containing a (FP29 gene and a 1×Flag affinity tag into a Mycobacterium smegmatis strain to obtain a recombinant Mycobacterium smegmatis strain; extracting a total protein solution of the recombinant Mycobacterium smegmatis strain, and subjecting the total protein solution of the recombinant Mycobacterium smegmatis strain to Flag tag affinity column chromatography purification to obtain a crude extract of the Mycobacterium smegmatis protein nanocage; and subjecting the crude extract of the Mycobacterium smegmatis protein nanocage to gel exclusion chromatography purification to obtain a pure product of the Mycobacterium smegmatis protein nanocage. In the present disclosure, the endogenously extracting the Mycobacterium smegmatis protein nanocage refers to extracting the Mycobacterium smegmatis protein nanocage from a recombinant Mycobacterium smegmatis strain containing a (FP29 gene and a 1×Flag affinity tag.

In the present disclosure, the recombinant Mycobacterium smegmatis strain is obtained by introducing a recombinant plasmid of Escherichia coli containing the (FP29 gene and the 1×Flag affinity tag into a Mycobacterium smegmatis strain. A purpose of introducing the recombinant plasmid of Escherichia coli containing the (FP29 gene and the 1×Flag affinity tag into the Mycobacterium smegmatis strain is to transform a genome of the wild-type Mycobacterium smegmatis strain, and to integrate the Flag affinity purification tag into the genome of the wild-type Mycobacterium smegmatis strain to obtain the recombinant Mycobacterium smegmatis strain.

In the present disclosure, a construction process of the recombinant plasmid includes: transferring the (′FP29 gene of the Mycobacterium smegmatis protein nanocage, an upstream nucleotide sequence of the (FP29 gene, a GGS-Linker, the 1×Flag affinity tag, a streptomycin selection marker, and a downstream nucleotide sequence of the (FP29 gene into a vector plasmid of Escherichia coli to obtain the recombinant plasmid of Escherichia coli. Optionally, the recombinant plasmid is constructed by PCR and homologous recombination. As an example, a preparation process of the recombinant plasmid of Escherichia coli includes: fusing encoding genes of the 1×Flag affinity tag and GGS-Linker at a C-terminal on the (FP29 gene of the Mycobacterium smegmatis protein nanocage, transferring the encoding genes onto the vector plasmid of Escherichia coli using homologous arms by homologous recombination, replicating and copying through Escherichia coli competent cells, and screening resistance markers to obtain a complete recombinant plasmid.

Optionally, the vector plasmid of Escherichia coli is pUC19; and the Escherichia coli competent cells are DH5α.

In the present disclosure, the (FP29 gene of the Mycobacterium smegmatis protein nanocage has a nucleotide sequence shown in SEQ ID NO: 1, specifically:

ATGAACAACCTCTATCGCGACCTCGCCCCGATCACCGAATCCGCTTGGG
CCGAGATCGAACTGGAGGCGACCCGCACGTTCAAGCGTCACATCGCCGG
ACGCCGGGTGGTCGACGTCAGCGGGCCCAACGGTCCGACGACCGCGAGC
GTCAGCACGGGTCATCTGCTCGACGTGAGCCCGCCCGGCGACGGCGTCA
TCGCGCATCTTCGCGATGCCAAACCGCTCGTGCGCCTGCGGGTGCCGTT
CACGGTGGCGCGCAGGGACATCGACGACGTCGAGCGCGGCTCGCAGGAC
TCCGACTGGGATCCGGTCAAGGACGCCGCCAAGAAGCTCGCGTTCGTCG
AGGACCGCGCGATCTTCGAGGGCTATGCCGCCGCGTCGATCGAGGGCAT
CCGCAGTTCCAGCTCCAACCCCGCGCTCGCACTGCCCGACGACGCCCGC
GAGATCCCCGACGTGATCGCCCAGGCCCTCTCCGAGCTGCGTCTGGCCG
GTGTCGACGGGCCCTACTCGGTGCTGCTCTCGGCCGAGACCTACACCAA
GGTCAGCGAGACCACCGCACACGGATATCCGATCCGCGAGCACATCAAC
CGCCTCGTCGACGGTGAGATCATCTGGGCGCCCGCGATCGACGGTGCGT
TCGTGTTGTCAACGCGCGGCGGTGATTTCGACCTGCAGCTCGGCACCGA
CGTGTCCATCGGCTACCTGTCCCATGACGCCGAGGTGGTCCACCTCTAC
ATGGAGGAGACCATGACGTTCCTGTGCTACACCGCTGAGGCCTCTGTCG
CGCTGACCCCC (795 bp).

In the present disclosure, the 1×Flag affinity tag has a nucleotide sequence shown in

SEQ ID NO: 2:
GATTACAAGGATGACGACGATAAG.

In the present disclosure, the GGS-Linker has an amino acid sequence: GGGGGTAGC.

In the present disclosure, the streptomycin selection marker has a nucleotide sequence shown in SEQ ID NO: 3, specifically:

TTGAATCGAACTAATATTTTTTTTGGTGAATCGCATTCTGACTGGTTGC
CTGTCAGAGGCGGAGAATCCGGTGATTTTGTTTTTCGACGTGGTGACGG
GCATGCCTTCGCGAAAATCGCACCTGCTTCCCGCCGCGGTGAGCTCGCT
GGAGAGCGTGACCGCCTCATTTGGCTCAAAGGTCGAGGTGTGGCTTGCC
CCGAGGTGATCAACTGGCAGGAGGAACAGGAGGGTGCATGCTTGGTGAT
AACGGCAATTCCGGGAGTACCGGCGGCTGATCTGTCTGGAGCGGATTTG
CTCAAAGCGTGGCCGTCAATGGGGCAGCAACTTGGCGCTGTTCACAGCC
TATCGGTTGATCAATGTCCGTTTGAGCGCAGGCTGTCGCGAATGTTCGG
ACGCGCCGTTGATGTGGTGTCCCGCAATGCCGTCAATCCCGACTTCTTA
CCGGACGAGGACAAGAGTACGCCGCAGCTCGATCTTTTGGCTCGTGTCG
AACGAGAGCTACCGGTGCGGCTCGACCAAGAGCGCACCGATATGGTTGT
TTGCCATGGTGATCCCTGCATGCCGAACTTCATGGTGGACCCTAAAACT
CTTCAATGCACGGGTCTGATCGACCTTGGGCGGCTCGGAACAGCAGATC
GCTATGCCGATTTGGCACTCATGATTGCTAACGCCGAAGAGAACTGGGC
AGCGCCAGATGAAGCAGAGCGCGCCTTCGCTGTCCTATTCAATGTATTG
GGGATCGAAGCCCCCGACCGCGAACGCCTTGCCTTCTATCTGCGATTGG
ACCCTCTGACTTGGGGTTGA (804 bp).

As an implementation method, a plasmid containing the streptomycin gene is used as a template to amplify 804 bp of the streptomycin gene using primers STR-F/STR-R. Preferably, the primers STR-F/STR-R are shown in SEQ ID NO: 10 and SEQ ID NO: 11.

As an implementation method, in the Escherichia coli recombinant plasmid, the upstream 806 bp nucleotide sequence of the CFP29 gene and the downstream 1,578 bp nucleotide sequence of the CFP29 gene are selected as upstream and downstream homologous arms, respectively. Using the wild-type Mycobacterium smegmatis strain as a template, primers Top-arm-F/Top-arm-R are used to amplify the upstream homologous arm of 806 bp; using the Mycobacterium smegmatis strain as a template, primers Bottom-arm-F/Bottom-arm-R are used to amplify the downstream homologous arm of 1,578 bp. Preferably, the primers Top-arm-F/Top-arm-R are shown in SEQ ID NO: 6 and SEQ ID NO: 7; the primers Bottom-arm-F/Bottom-arm-R are shown in SEQ ID NO: 8 and SEQ ID NO: 9.

In the present disclosure, the upstream 806 bp nucleotide sequence of the CFP29 gene is shown in SEQ ID NO: 4, specifically:

CCGAACTGCATCCGTTCCGCGAGATCGACGGTGGACGTCACCACGCACC
CGCGACTCCGGGCGATCTGCTGTTCCACCTGCGTGCCGAATCCATGGAC
GTGTGTTTCGAACTCGCGACCAAGCTCGTCGAGGCCATGTCCGGAGCCA
TCACGATCGTCGACGAGACGCACGGATTCCGGTTCTTCGACAACCGTGA
CCTCATGGGGTTCGTCGACGGTACCGAGAACCCGGACGGCAACCTCGCG
GTCGTCGCGACCCAGATCGGCGACGAGGACCCGGATTTCGCTGGCGGCT
GCTATGTGCACGTGCAGAAGTACCTGCACGACATGGCTTCCTGGAACTC
GCTGTCCGTCGAGGAGCAGGAACGCGTGATCGGCCGCACCAAGCTCGAC
GACATCGAGTTGGACGACGACGTCAAACCCGCCAATTCGCATGTGGCGC
TCAACGTCATCGAGGACGAGGACGGCAACGAGCTCAAGATAATTCGGCA
CAACATGCCGTTCGGCGAGATCGGCAAGGGCGAGTTCGGTACGTACTAC
ATCGGTTACTCGCGCACACCCAGCGTCACCGAACGCATGCTCGACAACA
TGTTCATCGGTGACCCGCCGGGCAACACCGACCGCATCCTGGATTTCTC
CACCGCCATCACCGGCGGACTGTTCTTCACCCCCACCGTCGACTTCCTC
GACGACCCACCACCTCTTCCGTCGGAGGACGACCGTGCCGAACCGGCTT
CGGCGCCTTCGGCCGACCCGGTCCACACCGACGGCTCACTCGGAATCGG
CAGCCTGAAAGGAACCCGCTGA.

In the present disclosure, the downstream 1,578 bp nucleotide sequence of the CFP29 gene is shown in SEQ ID NO: 5, specifically:

TGACCCCCTGACCCCGTAGCGGGTCACCCCGCACCGCGAGCGAGTCACC
CCGCACCGCGAGCGTGCGTGTCTGCTGCCCGGCACACCGCGAATCAGCA
GCACTCGGCGCACGCTCGTGGATGGCGAGAGTGCGCAGAGTGCTCAGCT
CGGCGGACTCAGAAGCGAAATCAGACCGACAGGATGGCGTCGAGCGCGG
AGTAGAACAGGCCCAGTCCGTCGTCGGACGGTCCGGTCAACGCCTCGGT
GGCGTGTTCGGGATGCGGCATGAGGCCGACGACGCGGCGATTCTCCGAG
CAGATGCCCGCGATGTCGCGCATCGACCCGTTGAGGTTCTCGCGATAGC
GGAAGACCACGCGGTCCTCGCCCTCGAGTTCGTCGAGCACGGACTCGGA
CGCCACGTAGCGCCCCTCGCCGGACTTCAGCGGGATCAGCAGGTCGGCA
CCGGTCTCGTAGCGCGTGGTCCACGCCGAGGTGTTGGACGCCACTTCGA
GCCACACGTCGCGGCAGACGAAGTGCAGACCCGCGTTGCGGGTCAGCGC
GCCGGGCAGCAGGCCGGCCTCGCACAGCACCTGGAAGCCGTTGCAGATG
CCGAGCACGGGCATGCCCTTGTTGGCGGCCTCGACCACCGAGCCCATCA
CGGGCGCGAACTTCGCGATGGCGCCGCACCGCAGGTAGTCCCCGTAGGA
GAAACCGCCGGGCACCACGACCGCGTCGACGCCGTGCAGATCCGCGTCG
GCGTGCCACAGGCTCACGGCCTCAGCACCCGCCAGACGGACCGCGCGGG
CCGCGTCGATGTCGTCCAGCGTGCCGGGGAACGTGATCACGCCCACACG
AGTGGTCATGCGTCCTCCCGGCTCACAGTGAAGTCCTCGATGACGGTGT
TCGCCAGGAGCGATTCCGCGATCTCGTTGAGGGTTTCGTCGGTCACGGA
ATCGTCGACCTCAAGCTCAAAACGCTTGCCCTGCCGAACGTCTGATATG
CCTTTGTGCCCGAGTCGGCCAAGCGCTCCCACGATGGCCTGACCTTGCG
GGTCGAGGATCTCAGCCTTGGGCATGACGTGCACAACCACCTTTGCCAC
GGCGTTCACTCTACCGGCACGATCCCAGGTAGGCGTCACACAGTGGTGC
GGCCATCGCGTCGAGCACCTGGCTATCGTCCACCGACGGCCACGGCACC
TGCGGCGGGGTCGTCGACCACAGCGCGAGCTCCACGACGGTGCTGTTGT
TGGGGTCGGCGAGCAAATACTGGCGCAGAACCCGGTCGCCGAAGGCGAT
CACCCCGGCCAGCCGGCCCGGTTCGTCGGTGGTGAGCGACGGTGAGCTG
TCGGGCGCCACCAGCTGACACGCCCGGATCGCGGCGGCCGACGCGCTCA
CGGCCTCCATCGCGAGCTGCCCGCCCCGCCAGGTCTCGCCGCGCCAGTG
GATGATCTGCGCGGAGAGCTGCCACTGCCCCACCGGCCCCGGGCCACCT
CGGCCCGCGCGCTCACCGCATAGTGCCGCGGATCGTCGGGCAGCAGCGG
CAGCGCGCACTCCTGTTCGAACCGGAACCGCGGCGACACCGTCGTCACG
GCCAAACCGG.

In the present disclosure, a pUC19 linearized vector of about 2,600 bp is amplified using the Escherichia coli vector plasmid pUC19 as a template and primers pUC-F/pUC-R. A purpose of amplifying the pUC19 linearized vector of about 2,600 bp is to provide the linearized vector for recombination and ligation with a subsequent target fragment. The primers pUC-F/pUC-R are shown in SEQ ID NO: 13 and SEQ ID NO: 14.

As an implementable embodiment, the PCR-amplified and purified CFP29 gene of Mycobacterium smegmatis protein nanocage, the upstream nucleotide sequence of the CFP29 gene, the downstream nucleotide sequence of the CFP29 gene, the GGS-Linker, the 1×Flag affinity tag, the streptomycin selection marker, and the pUC19 linearized vector fragment are ligated using a commercial homologous recombinase according to the instructions. The commercial homologous recombinase can be obtained from commercial channels without limitation.

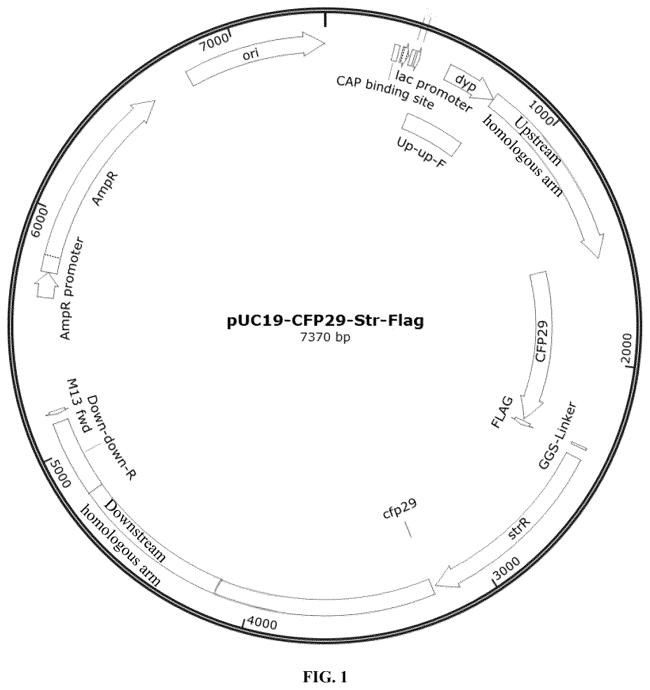

Preferably, the Escherichia coli recombinant plasmid is pUC19-CFP29-Str-Flag, and the recombinant plasmid has a map shown in FIG. 1.

In the present disclosure, the construction process of the recombinant Mycobacterium smegmatis strain further includes: electroporating the Escherichia coli recombinant plasmid pUC19-CFP29-Str-Flag into competent cells of Mycobacterium smegmatis, and using streptomycin as a selection marker to obtain the recombinant Mycobacterium smegmatis strain with a 1×Flag affinity purification tag. As an example, the competent cells of Mycobacterium smegmatis are Mycobacterium smegmatis competent cells pJV53. In the examples, the plasmid pJV53 is cited from Van Kessel J C, Hatfull G F. Recombineering in Mycobacterium tuberculosis. Nat Methods. 2007 February; 4 (2): 147-52. A preparation process of the Mycobacterium smegmatis competent cells pJV53 includes: preparing wild-type Mycobacterium smegmatis mc²155 competent cells, and constructing the competent cells pJV53 based on the wild-type Mycobacterium smegmatis mc²155 competent cells. Preferably, a wild-type Mycobacterium smegmatis mc²155 strain is purchased from the American Type Culture Collection (ATCC), with a deposit number of ATCC No. 700084. pJV53 can express recombinase, such that the streptomycin-resistant (FP29 gene and GGS and Flag tags in the Escherichia coli recombinant plasmid pUC19-CFP29-Str-Flag can be integrated into the Mycobacterium smegmatis genome by homologous recombination replacement to obtain the recombinant Mycobacterium smegmatis strain.

In the present disclosure, the recombinant Mycobacterium smegmatis strain is cultured and its bacterial cells are collected. As an example, the recombinant strain is cultured in an LB broth with shaking at preferably 36° C. to 38° C. under a shaking frequency of 200 rpm to 250 rpm for 4 d to 6 d.

As an example, a process of extracting the total protein solution of the recombinant Mycobacterium smegmatis strain includes: resuspending the recombinant Mycobacterium smegmatis bacterial cell with a resuspension reagent, conducting high-pressure cell disruption, and collecting a supernatant obtained by centrifugation to obtain the total protein solution. The resuspension reagent preferably uses a basic buffer. The basic buffer reagent includes preferably 3-(N-morpholino) propanesulfonic acid, sodium chloride, and PMSF. Specifically, the basic buffer includes 20 mmol/L of the 3-(N-morpholino) propanesulfonic acid, 100 mmol/L of the sodium chloride, and 1 mmol/L of the PMSF, and has a pH value of 7.4. When the bacterial cell of the recombinant Mycobacterium smegmatis strain is resuspended, the bacterial cell and the resuspension reagent are at a mass-to-volume ratio of preferably 1:(5-8).

In the present disclosure, after the high-pressure cell disruption of the resuspended bacterial cell is completed, the supernatant is collected after centrifugation at 12,000 rpm to 15,000 rpm for 10 min to 20 min. 1% to 3% of a detergent POELE (Brij35) by weight percentage (W/V) is added into the supernatant, shaken in an ice bath for 2 h to 3 h, centrifuged at 12,000 rpm to 15,000 rpm for 30 min to 60 min to collect a supernatant, namely the total protein solution of Mycobacterium smegmatis.

The method includes: subjecting the total protein solution of the recombinant Mycobacterium smegmatis strain to Flag tag affinity column chromatography purification to obtain a crude extract of the Mycobacterium smegmatis protein nanocage; and subjecting the crude extract of the Mycobacterium smegmatis protein nanocage to gel exclusion chromatography purification to obtain a pure product of the Mycobacterium smegmatis protein nanocage.

In the present disclosure, the Flag tag affinity column chromatography purification is preferably conducted on a chromatographic column filled with an Anti-Flag filler. The Flag tag affinity column chromatography purification preferably includes impurity elution and elution that are conducted in sequence. In the present disclosure, an impurity eluent used for the impurity elution preferably includes 3-(N-morpholino) propanesulfonic acid (MOPS), NaCl, and a detergent. In the impurity eluent, the 3-(N-morpholino) propanesulfonic acid (MOPS) has a molar concentration of preferably 20 mmol/L; the NaCl has a molar concentration of preferably 100 mmol/L; and the detergent has a mass percentage (W/V) of preferably 0.05%. The detergent is preferably POELE (Brij35). The impurity eluent has a pH value of preferably of 7.4. The impurity eluent has a volume preferably 5 to 10 times that of the chromatographic column filled with the Anti-Flag filler. The impurity elution is intended to reduce the enrichment of non-specific impurity proteins.

In the present disclosure, the elution is preferably competitive elution. An eluent used for the elution preferably includes 3-(N-morpholino) propanesulfonic acid (MOPS), NaCl, POELE (Brij35), and 1×Flag peptide. In the eluent, the 3-(N-morpholino) propanesulfonic acid (MOPS) has a molar concentration of preferably 20 mmol/L; the NaCl has a molar concentration of preferably 100 mmol/L; the detergent has a mass percentage (W/V) of preferably 0.05%, and the detergent is preferably POELE (Brij35); and the 1×Flag peptide has a mass concentration of preferably 150 μg/mL. The eluent has a pH value of preferably 7.4. The eluent has a volume preferably 5 to 10 times that of the chromatographic column filled with the Anti-Flag filler. The 1×Flag peptide has an amino acid sequence: DYKDDDDK. In the present disclosure, the elution is to obtain purer Mycobacterium smegmatis protein nanocage than the initial crude extract.

The Flag tag affinity column chromatography purification further includes concentrating a resulting eluate; the concentration is preferably centrifugal concentration at 3,200 rpm, and the centrifugal concentration is preferably conducted in a concentration tube with a molecular-weight cutoff of 100 kDa. There are no special requirements for a specific operation of the centrifugal concentration, as long as it can meet the subsequent gel exclusion chromatography purification. The ultimate goal is to concentrate a volume of the eluate to not more than 1 mL.

In the present disclosure, a concentrated sample of the Mycobacterium smegmatis protein nanocage is preferably subjected to the gel exclusion chromatography purification to finally obtain the pure product of the Mycobacterium smegmatis protein nanocage. The gel exclusion chromatography purification is preferably conducted on a chromatographic column of Superose 6 increase 10/300 gel exclusion. The gel exclusion chromatography purification is preferably conducted using an equilibrium buffer. The equilibrium buffer used for the gel exclusion chromatography purification preferably includes 3-(N-morpholino) propanesulfonic acid (MOPS) and NaCl; the 3-(N-morpholino) propanesulfonic acid (MOPS) has a molar concentration of preferably 20 mmol/L; the NaCl has a molar concentration of preferably 100 mmol/L; and the equilibrium buffer has a pH value of preferably 7.4. The equilibrium buffer has a volume of preferably 25 mL. The equilibrium buffer is added to obtain the pure product of the Mycobacterium smegmatis protein nanocage.

In the present disclosure, a 1×Flag affinity purification tag is inserted into a C-terminal on a gene of the Mycobacterium smegmatis protein nanocage by homologous recombination, and then Flag tag affinity column chromatography purification and gel exclusion chromatography purification are combined to obtain the pure product of the Mycobacterium smegmatis protein nanocage. The extracted Mycobacterium smegmatis background protein nanocage obtained after genome modification has high yield, excellent purity, and stable properties. Moreover, the method has simple steps, high sample purity, and short purification cycle, as well as low requirements for experimental instruments and equipment, thus greatly reducing time and financial costs.

The technical solutions of the present disclosure will be clearly and completely described below with reference to the examples of the present disclosure. Apparently, the described embodiments are merely some rather than all of the embodiments of the present disclosure. All other examples obtained by a person of ordinary skill in the art based on the examples of the present disclosure without creative efforts shall fall within the protection scope of the present disclosure.

In the example of the present disclosure, the chromatographic column for the Flag tag affinity column chromatography purification is a commercial product, with product information of Anti-DYKDDDDK G1 Affinity Resin (Genscript Biotechnology Co., Ltd.); and the chromatographic column for the gel exclusion chromatography purification is a commercial product, with product information of Superose6 increase10/300 (GE Healthcare). Unless otherwise specified, other reagents and materials used in the examples can be obtained from commercial sources.

Unless otherwise specified, all experimental methods in the following examples are conventional methods.

### Example 1 Construction of a Recombinant Strain Expressing the Mycobacterium smegmatis Protein Nanocage

1. Construction of Mycobacterium smegmatis Competent Cells Containing Plasmid pJV53
(1) Construction of Mycobacterium smegmatis glycerol stock containing plasmid pJV53

The wild-type Mycobacterium smegmatis mc²155 competent cells were prepared, and the competent cells pJV53 were constructed based on the competent cells. The wild-type Mycobacterium smegmatis mc²155 competent cells (purchased from the ATCC, with a deposit number of ATCC No.?) were thawed on ice, and 1 μg of a correctly sequenced pJV53 plasmid (cited from Van Kessel J C, Hatfull G F. Recombineering in Mycobacterium tuberculosis. Nat Methods. 2007 February; 4 (2): 147-52) was added in a clean bench, mixed well, and then placed in an ice bath for 30 min. An electroporation instrument program was set as follows: voltage 2,500 V, time 5.7 ms, and electroporation cup thickness 2 mm, and electroporation was conducted to allow the plasmid to enter the cells. After the electric shock, the electroporation cup was quickly placed on ice for about 5 min, and 800 μL of a sterilized LBT broth was added, and the cells were revived at 180 rpm and 37° C. After 5 h, the cells were centrifuged at 4,000 rpm for 10 min, 1 mL of a supernatant was discarded, and the remaining supernatant was mixed well with the pellet, and then spread evenly with a spreading rod on a solid medium containing 50 μg/mL kanamycin and 40 μg/mL carbenicillin sodium. The cells were incubated at 37° C. in an incubator for three days and a single colony strain was selected.

The LB solid medium included: 10 g tryptone, 10 g sodium chloride (NaCl), 5 g yeast extract, and 15 g agar were diluted with water to 1 L, sterilized with high-pressure steam at 121° C. for 30 min, cooled to 50° C., added with antibiotics, and poured into a petri dish for later use. Each dish has about 20 mL of a solid medium liquid.

The LB broth included: 10 g tryptone, 10 g sodium chloride (NaCl), 5 g yeast extract, 15 g agar, and 1 g Tween 80 were diluted with water to 1 L, sterilized with high-pressure steam at 121° C. for 30 min.
(2) 5 μL of 50 mg/mL kanamycin, 5 μL of 40 mg/mL carbenicillin sodium were mixed well in 5 mL of medium, a single colony was selected from the cultured plate, and subjected to shaking culture at 37° C. and 220 rpm for 48 h. Bacterial liquid expansion: 200 μL of 50 mg/mL kanamycin and 200 μL of 40 mg/mL carbenicillin sodium were mixed well in 200 mL LBT broth, and 2 mL of cultured Mycobacterium smegmatis bacterial solution containing plasmid pJV53 (LBT broth: bacterial solution in a volume ratio=100:1) was added to allow shaking culture at 37° C. and 220 rpm for about 6 h to 10 h until a bacterial concentration reached an OD600 value of about 0.6 to 0.8. 1 mL of 40% (w/v) acetamide solution was added to a resulting bacterial solution to allow shaking culture at 37° C. and 220 rpm for about 4 h. The bacterial solution was treated in an ice bath for 2.5 h.
(3) The bacterial solution was centrifuged at 3,200 rpm for 10 min at 4° C., and a supernatant was discarded. The bacterial cells were washed under sterile conditions, and 30 mL of sterilized 10% glycerol pre-cooled on ice was added to gently suspend the bacterial cells. The bacterial solution was centrifuged at 3,200 rpm, 10 min, 4° C. and a supernatant was discarded. The above washing steps were repeated three times. 8 mL of sterilized 10% glycerol in an ice bath was added into the bacterial cells after washing three times, the bacterial cells were suspended by gently pipetting, and 180 μL of a resulting bacterial suspension was dispensed into each tube to obtain the Mycobacterium smegmatis competent cells containing the pJV53 plasmid. The competent cells were quickly frozen in liquid nitrogen and stored at −80° C.
   2. Construction of a Recombinant Strain Expressing the Mycobacterium smegmatis Protein Nanocage

A recombinant plasmid pUC19-CFP29-Str-Flag was constructed by PCR and homologous recombination. A preparation process of the recombinant plasmid pUC19-CFP29-Str-Flag included: a pUC19 linearized vector of about 2,600 bp was amplified using primers pUC-F/pUC-R (as shown in SEQ ID NO: 12 and SEQ ID NO: 13) with pUC19 plasmid as a template. The 804 bp streptomycin gene (as shown in SEQ ID NO: 3) was amplified using primers STR-F/STR-R (as shown in SEQ ID NO: 10 and SEQ ID NO: 11) with a plasmid containing the streptomycin gene as a template. The (FP29 gene of Mycobacterium smegmatis protein nanocage (as shown in SEQ ID NO: 1), the upstream 806 bp nucleotide sequence of the CFP29 gene (as shown in SEQ ID NO: 4), and the downstream 1,578 bp nucleotide sequence of the CFP29 gene (as shown in SEQ ID NO: 5) were amplified by PCR. The GGS-Linker was amplified using a plasmid containing a GGS-Linker gene (GGGGGTAGC) as a template, and the 1×Flag affinity tag was amplified using a plasmid containing a 1×Flag affinity tag gene (as shown in SEQ ID NO: 2) as a template.

The pUC19 linearized vector and the CFP29 gene of the Mycobacterium smegmatis protein nanocage (as shown in SEQ ID NO: 1), the upstream 806 bp nucleotide sequence of the CFP29 gene (as shown in SEQ ID NO: 4), GGS-Linker (GGGGGTAGC), 1×Flag affinity tag (as shown in SEQ ID NO: 2), streptomycin selection marker (as shown in SEQ ID NO: 3), and the downstream 1,578 bp nucleotide sequence of the CFP29 gene (as shown in SEQ ID NO: 5) were ligated using a commercial homologous recombinase according to instructions to obtain the recombinant plasmid pUC19-CFP29-Str-Flag.

The constructed recombinant plasmid pUC19-CFP29-Str-Flag was sent to a sequencing company for sequencing. The plasmid after successful sequencing was transformed into the E. coli DH5a strain, and the recombinant plasmid pUC19-CFP29-Str-Flag was obtained by sequencing with a pUC19 universal primers using ampicillin as a resistance selection marker, with a map shown in FIG. 1.

The primers used to construct the recombinant plasmid pUC19-CFP29-Str-Flag and the recombinant strain were shown in Table 1, and the amplification program was shown in Table 2.

Table 1
Primers used to construct recombinant plasmid pUC19-CFP29-Str-Flag and
recombinant E. coli strain

| | | Number |
|---|---|---|
| Primer name | Primer sequence (5′ to 3′) | of bases |
| Top-arm-F | TTGCATGCCTGCAGGTCGACCCGAACTGCATCCGTTCCGCGAGAT | 45 |
| (SEQ ID NO: 6) | | |
| Top-arm-R | TTCTCACTTATCGTCGTCATCCTTGTAATCGCTACCCCCGGGGGTCA | 64 |
| (SEQ ID NO: 7) | GCGCGACAGAGGCCTCA | |
| Bottom-arm-F | TGGGGTTGAACTAGTGATCGATATCTGACCCCCTGACCCCGTAGCG | 49 |
| (SEQ ID NO: 8) | GGT | |
| Bottom-arm-R | GTACCCGGGGATCCTCTAGACCGGTTTGGCCGTGACGACG | 40 |
| (SEQ ID NO: 9) | | |
| STR-F | ATTACAAGGATGACGACGATAAGTGAGAATTCtagaggtccgctTTGA | 59 |
| (SEQ ID NO: 10) | ATCGAACTAAT | |
| STR-R | GATATCGATCACTAGTTCAACCCCAAGTCAGAGGGTCCAATCG | 43 |
| (SEQ ID NO: 11) | | |
| pUC-F | TCTAGAGGATCCCCGGGTACCGAGC | 25 |
| (SEQ ID NO: 12) | | |
| pUC-R | GTCGACCTGCAGGCATGCAAGC | 22 |
| (SEQ ID NO: 13) | | |

TABLE 2
Program settings for amplifying each target fragment

| Target fragment | Annealing | Extension |
|---|---|---|
| Upstream homologous arm | 70° C., 15 s | 72° C., 1 min 20 s |
| Downstream homologous arm | 62° C., 15 s | 72° C., 1 min 30 s |
| Streptomycin gene | 70° C., 15 s | 72° C., 40 s |
| pUC vector fragment | 59° C., 15 s | 72° C., 1 min 40 s |

NOTE: the amplification of all target fragments included: initial denaturation at 95° C. for 10 min; denaturation at 95° C. for 15 s; 35 cycles; supplementary extension at 72° C. for 10 min; storage at 4° C.; where only the annealing and extension program settings were different.

The prepared Mycobacterium smegmatis competent cells containing pJV53 plasmid were thawed on ice, and 1 μg of correctly sequenced recombinant plasmid pUC19-CFP29-Str-Flag was added in a clean bench, mixed well, ice-bathed for 30 min, and transferred into a 2 mm thick electroporation cup that had been ice-bathed in advance. An electroporation instrument program was set as follows: voltage 2,500 V, time 5.7 ms, and electroporation cup thickness 2 mm, and electroporation was conducted to allow the plasmid to enter the cells. After the electric shock, the electroporation cup was quickly placed on ice for about 5 min, and 800 μL of a sterilized LBT broth was added, and the cells were revived at 180 rpm and 37° C. After 5 h, the cells were centrifuged at 4,000 rpm for 10 min, 1 mL of a supernatant was discarded, and the remaining supernatant was mixed well with the pellet, and then spread evenly with a spreading rod on a solid medium containing 40 μg/mL carbenicillin sodium and 25 μg/mL streptomycin, cultured in a 37° C. constant-temperature incubator for three days, and selected sites were screened to obtain a recombinant Mycobacterium smegmatis strain (pJV53-CFP29-Str-Flag). When obvious colonies appeared on the plate, 5 μL of 40 mg/ml carbenicillin sodium and 2.5 μL of 50 mg/mL streptomycin were mixed well in 5 mL of LBT broth, a single colony of the recombinant Mycobacterium smegmatis strain was selected from the cultured plate, and subjected to shaking culture at 37° C. and 220 rpm for 48 h.

An upstream primer Up-up-F was designed 1,600 bp upstream and a downstream primer Down-down-R was designed 1,578 bp downstream of the CFP29 target fragment. The upstream primer Up-up-F (SEQ ID NO: 14) was: CCATTTGTGCCCGCACCACAGCCTCA; downstream primer Down-down-R (SEQ ID was: NO: 15) GCCAAAAGGCACGAATGTCCGAAATGGGTCAGTT. Primers Up-up-F/Down-down-R were used to conduct PCR on the recombinant Mycobacterium smegmatis strain as a template, while the wild-type Mycobacterium smegmatis strain was used as a negative control to detect the recombinant plasmid pJV53-CFP29-Str-Flag. If a fragment of about 4500 bp was amplified, it was confirmed that the resistance gene as well as GGS and Flag tags were integrated into the genome.

The purified PCR products were sequenced to confirm the genome integrity of the recombinant strain. The recombinant Mycobacterium smegmatis strain with completely correct sequencing results was preserved by mixing 500 μL of 50% glycerol with 500 μL of the bacterial solution. After this process, the final recombinant strain, namely the recombinant Mycobacterium smegmatis strain could be obtained. The primers used for amplification were shown in Table 3.

TABLE 3
Sequencing primers used to detect genome integrity of recombinant
Mycobacterium smegmatis strain

| Primer name | Primer sequence (5′ to 3') | Number of bases |
|---|---|---|
| Sequence 1 (SEQ ID NO: 16) | CTCACATGTTCTTTCCTGCG | 20 |
| Sequence 2 (SEQ ID NO: 17) | ATCGGCAAGGGCGAGTTCGG | 20 |
| Sequence 3 (SEQ ID NO: 18) | AGCACATCAACCGCCTCGTC | 20 |
| Sequence 4 (SEQ ID NO: 19) | GATCGACCTTGGGCGGCTCG | 20 |
| Sequence 5 (SEQ ID NO: 20) | GCACCGCAGGTAGTCCCCGT | 20 |
| Sequence 6 (SEQ ID NO: 21) | GAGGTGGTGGGTCGTCGAGG | 20 |
| Sequence 7 (SEQ ID NO: 22) | CCGCCGGTACTCCCGGAATT | 20 |
| Sequence 8 (SEQ ID NO: 23) | CACGTCATGCCCAAGGCTGA | 20 |

### Example 2 Extraction and Purification of Mycobacterium smegmatis Protein Nanocage

1. Extraction of Total Protein Solution of Mycobacterium smegmatis
(1) Culture of Recombinant Mycobacterium smegmatis Strain

50 μL of 40 mg/mL carbenicillin sodium and 25 μL of 50 mg/mL streptomycin were added into 50 mL of LBT broth and mixed well, and 1 mL of a glycerol stock of the recombinant Mycobacterium smegmatis strain preserved in Example 1 was added, and a resulting mixture was cultured at 37° C. and 220 rpm for about 30 h to allow activation. The culture was expanded to 1 L of medium, each 1 L of which contained 1 mL of 40 mg/mL carbenicillin sodium and 500 μL of 50 mg/mL streptomycin, and then 5 mL of activated recombinant Mycobacterium smegmatis strain was added and cultured at 37° C. and 220 rpm for 5 d. The bacterial solution was centrifuged at 4,000 rpm for 15 min to collect the bacterial cells, the supernatant was discarded, and the pellet was retained at −20° C. for later use.

(2) Preparation of Total Protein Solution of Recombinant Mycobacterium smegmatis Strain

The bacterial cells of recombinant Mycobacterium smegmatis strain were resuspended in a basic buffer at dosage ratio of 1:6; where the basic buffer included: 20 mmol/L 3-(N-morpholino) propanesulfonic acid (MOPS), 100 mmol/L sodium chloride, and 1 mmol/L PMSF, pH=7.4. After being mixed evenly, the bacterial cells were lysed using a high-pressure homogenizer, with the setting conditions: 4° C., 1,000 bar, and 5 cycles of lysis. The cells were centrifuged at 14,000 rpm for 15 min at 4° C., and the supernatant was collected for later use.

10% by weight (W/V) of Brij35 was added into the supernatant, such that a mass percentage (W/V) of Brij35 in the entire incubation system reached 1%. The bacterial cells were incubated in a shaking incubator at 4° C. for 2 h. A supernatant was collected by centrifugation at 14,000 rpm for 40 min at 4° C. to obtain the total protein solution of the recombinant Mycobacterium smegmatis strain.

2. Purification of Mycobacterium smegmatis Protein Nanocage

20 mL of impurity eluent was added to a gravity column filled with Anti-Flag filler for equilibration, where the impurity eluent (pH=7.4) included: 20 mmol/L 3-(N-morpholino) propanesulfonic acid (MOPS), 100 mmol/L sodium chloride, and 0.05% Brij35 by weight percentage (W/V). After liquid flowed out naturally, the column was set aside, the column should be prevented from being completely drained, and the filler should always be kept moist. A collected total protein crude extract naturally flowed through the gravity column and was repeatedly hung on the column 4 to 5 times. After the crude protein extract had flowed away, the gravity column was cleaned with an appropriate amount of the impurity eluent, and Coomassie Brilliant Blue dye (G250) was added to determine the removal of impurity proteins. When Coomassie Brilliant Blue (G250) no longer turned blue, the impurity removal was completed. The protein was eluted into a concentrator tube with a molecular-weight cutoff of 100 KDa using 20 mL of an eluent, and a resulting eluate was concentrated; where the eluent (pH=7.4) included: 20 mmol/L 3-(N-morpholino) propanesulfonic acid (MOPS), 100 mmol/L sodium chloride, 0.05% Brij35 by weight (W/V), and 1×Flag peptide with a mass concentration of 150 μg/mL. A volume of the eluate was concentrated to not more than 1 mL by centrifugation at 3,200 rpm for 30 min at 4° C. to obtain the Mycobacterium smegmatis protein nanocage with high purity.

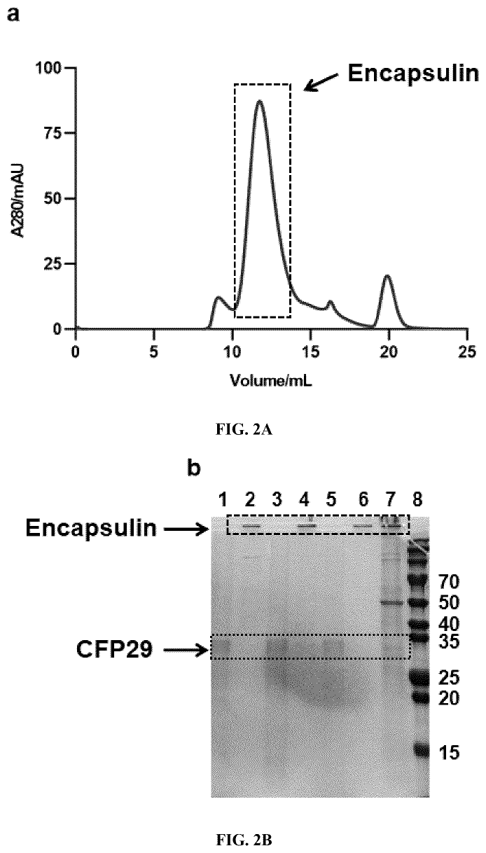

A chromatographic column of Superose6 increase 10/300 gel exclusion chromatography was equilibrated with 1 column volume (25 mL) of equilibration buffer (pH=7.4), including: 20 mmol/L 3-(N-morpholino) propanesulfonic acid (MOPS) and 100 mmol/L sodium chloride, the crude extract of Mycobacterium smegmatis protein nanocage was loaded on the chromatographic column of Superose6 increase 10/300 gel exclusion chromatography, and eluted with 25 mL of equilibrium buffer. The results were shown in FIG. 2A. FIG. 2A showed an elution curve of Superose 6 10/300 GL, where the horizontal axis represented an elution volume of the chromatographic column, in mL; the vertical axis represented an absorbance of the UV spectrophotometer at 280 nm, in mAU. As shown in FIG. 2A, the Mycobacterium smegmatis protein nanocage exhibited a single characteristic peak at 12 mL to 12.5 mL. The single characteristic peak could determine that the sample at characteristic peak position was the Mycobacterium smegmatis protein nanocage extracted in the present disclosure.

The samples at characteristic peak position were collected to obtain a Mycobacterium smegmatis protein nanocage solution. The Mycobacterium smegmatis protein nanocage solution was subjected to 12% SDS-PAGE electrophoresis detection, and the detection results were shown in FIG. 2B. FIG. 2B showed the SDS-PAGE (12%) electrophoresis detection results of the Mycobacterium smegmatis protein nanocage solution, where:
Lanes 1-3-5: concentrated sample (fraction) Nov. 13, 2017, heated at 100° C. for 30 min after sample preparation and then loaded;
Lanes 2-4-6: concentrated sample (fraction) Nov. 13, 2017, loaded directly without heating after sample preparation;
Lane 7: concentrated sample from molecular sieve; and
Lane 8: Protein Marker: Protein Ladder.

The results in FIG. 2B showed that the Mycobacterium smegmatis protein nanocage had stable protein properties.

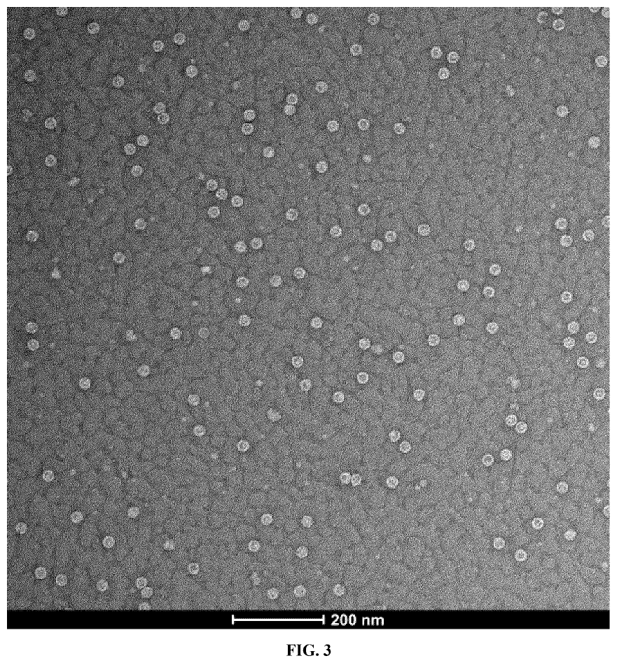

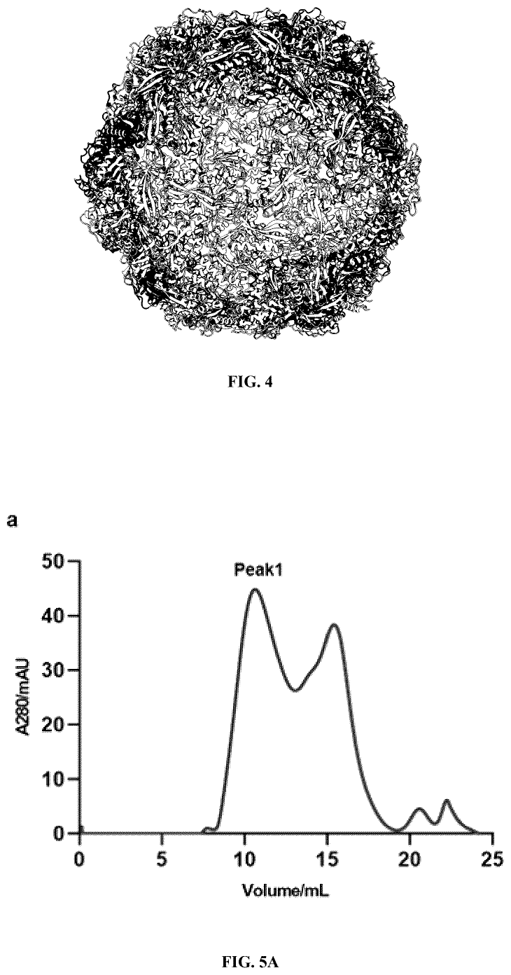

FIG. 3 showed a negative staining electron microscopy result of the Mycobacterium smegmatis protein nanocage. FIG. 3 showed that the sample had uniform and pure particle size. The structure of the Mycobacterium smegmatis protein nanocage (PDB: 7BOJ) was shown in FIG. 4. The Mycobacterium smegmatis protein nanocage molecule was composed of 60 identical subunit copies and had a regular icosahedral structure with a resolution of 2.5 Å.

### Comparative Example

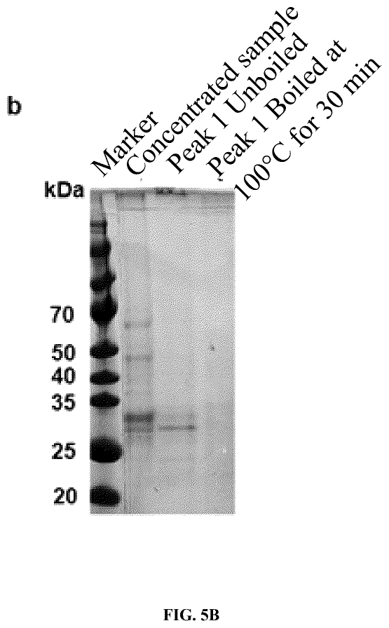

The Mycobacterium smegmatis protein nanocage in the wild-type strain (with its own His tag) was extracted using the method of Example 2, and the results were shown in FIGS. 5A-5B.

FIG. 5A was the elution curve of the wild-type and His-tagged purified Mycobacterium smegmatis protein nanocage by gel filtration chromatography of Superose 6 increase 10/300 GL. The A280/mAU on the ordinate was proportional to the total amount of protein. The purity could be reflected by whether the elution peak was single or a ratio of the non-target protein peak to the target protein peak height. The closer the non-target protein peak height was to the target protein peak height, the lower the protein purity was, and vice versa. FIG. 5A indicated that the sample directly purified from wild-type Mycobacterium smegmatis had a higher non-target protein peak, proving that there was a poor purity of the protein nanocage.

FIG. 5B was an SDS gel electrophoresis diagram, showing the purity of the target protein based on whether the band was single or not. It was determined that the protein nanocage purified by this method was mixed with a large number of non-target proteins. According to the results of FIG. 5B, its purity needed to be improved.

As shown above, FIG. 2A and FIG. 5A were the elution curves of the Mycobacterium smegmatis protein nanocage purified from recombinant Mycobacterium smegmatis strain (protein nanocage obtained by background purification) and the Mycobacterium smegmatis protein nanocage purified from wild-type Mycobacterium smegmatis strain (with its own His tag) by the gel filtration chromatography of Superose 6 increase 10/300 GL, respectively. Compared with that in FIG. 5A, the peak height of the non-target protein in FIG. 2A was farther away from the peak height of the target protein, indicating that there was a higher protein purity. After further inspection by SDS-PAGE, the gel images of FIG. 2B and FIG. 5B showed that the protein nanocage bands obtained by background purification were clearer and more single than those of the wild-type natural expression bands. In summary, the protein nanocage obtained in the present disclosure had high yield and excellent purity.

In the present disclosure, the method has simple operations, convenience, and short experimental cycle during construction and purification. Moreover, the Mycobacterium smegmatis background protein nanocage shows high yield, excellent purity, and stable properties compared to the previous method. The above characteristics are of great significance for the subsequent study of the structure and function of Mycobacterium smegmatis protein nanocage and advantages thereof in drug delivery.

Although the above example has described the present disclosure in detail, it is only a part of, not all of, the examples of the present disclosure. Other examples may also be obtained by persons based on the example without creative efforts, and all of these examples shall fall within the protection scope of the present disclosure.

What is claimed is:

1. A method for endogenously extracting a Mycobacterium smegmatis protein nanocage, comprising the following steps:
introducing a recombinant plasmid containing a CFP29 gene and a 1×Flag affinity tag into a Mycobacterium smegmatis strain to obtain a recombinant Mycobacterium smegmatis strain; extracting a total protein solution of the recombinant Mycobacterium smegmatis strain, and subjecting the total protein solution of the recombinant Mycobacterium smegmatis strain to Flag tag affinity column chromatography purification to obtain a crude extract of the Mycobacterium smegmatis protein nanocage; and subjecting the crude extract of the Mycobacterium smegmatis protein nanocage to gel exclusion chromatography purification to obtain a pure product of the Mycobacterium smegmatis protein nanocage.

2. The method according to claim 1, wherein the recombinant plasmid is pUC19-CFP29-Str-Flag.

3. The method according to claim 1, wherein a construction process of the recombinant plasmid comprises: transferring the CFP29 gene, an upstream 806 bp nucleotide sequence of the CFP29 gene, a GGS-Linker, the 1×Flag affinity tag, a streptomycin selection marker, and a downstream 1,578 bp nucleotide sequence of the CFP29 gene into a vector plasmid.

4. The method according to claim 3, wherein the upstream 806 bp nucleotide sequence of the CFP29 gene is shown in SEQ ID NO: 5, and the downstream 1,578 bp nucleotide sequence of the CFP29 gene is shown in SEQ ID NO: 6.

5. The method according to claim 1, wherein the Flag tag affinity column chromatography purification is conducted on a chromatographic column filled with an Anti-Flag filler; and the gel exclusion chromatography purification is conducted on a chromatographic column of Superose 6 increase 10/300 gel exclusion.

6. The method according to claim 1, wherein the Flag tag affinity column chromatography purification comprises impurity elution and elution that are conducted in sequence; an impurity eluent for the impurity elution comprises 3-(N-morpholino) propanesulfonic acid, NaCl, and a detergent; and a eluent for the elution comprises the 3-(N-morpholino) propanesulfonic acid, the NaCl, and a 1×Flag peptide.

7. The method according to claim 6, wherein the detergent comprises polyoxyethylene lauryl ether (POELE).

8. The method according to claim 1, wherein an equilibrium buffer for the gel exclusion chromatography purification comprises 3-(N-morpholino) propanesulfonic acid and NaCl.

9. The method according to claim 8, wherein the 3-(N-morpholino) propanesulfonic acid has a molar concentration of 20 mmol/L to 25 mmol/L and the NaCl has a molar concentration of 100 mmol/L to 110 mmol/L in the equilibrium buffer.

10. The method according to claim 1, wherein a process of extracting the total protein solution of the recombinant Mycobacterium smegmatis strain comprises: culturing the recombinant Mycobacterium smegmatis strain, collecting a resulting recombinant Mycobacterium smegmatis bacterial cell, resuspending the recombinant Mycobacterium smegmatis bacterial cell with a resuspension reagent, conducting high-pressure cell disruption, and collecting a supernatant obtained by centrifugation to obtain the total protein solution.

11. The method according to claim 2, wherein a construction process of the recombinant plasmid comprises: transferring the CFP29 gene, an upstream 806 bp nucleotide sequence of the CFP29 gene, a GGS-Linker, the 1×Flag affinity tag, a streptomycin selection marker, and a downstream 1,578 bp nucleotide sequence of the CFP29 gene into a vector plasmid.

12. The method according to claim 11, wherein the upstream 806 bp nucleotide sequence of the CFP29 gene is shown in SEQ ID NO: 5, and the downstream 1,578 bp nucleotide sequence of the CFP29 gene is shown in SEQ ID NO: 6.


SEQUENCE LISTING (23 sequences)

SEQ ID NO: 1 - DNA, 795 bp, synthetic construct
  source 1..795: mol_type = other DNA
atgaacaacctctatcgcgacctcgccccgatcaccgaatccgcttgggccgagatcgaactggaggcgacccgcacgttcaagcgtcacatcgccggacgccgggtggtcgacgtcagcgggcccaacggtccgacgaccgcgagcgtcagcacgggtcatctgctcgacgtgagcccgcccggcgacggcgtcatcgcgcatcttcgcgatgccaaaccgctcgtgcgcctgcgggtgccgttcacggtggcgcgcagggacatcgacgacgtcgagcgcggctcgcaggactccgactgggatccggtcaaggacgccgccaagaagctcgcgttcgtcgaggaccgcgcgatcttcgagggctatgccgccgcgtcgatcgagggcatccgcagttccagctccaaccccgcgctcgcactgcccgacgacgcccgcgagatccccgacgtgatcgcccaggccctctccgagctgcgtctggccggtgtcgacgggccctactcggtgctgctctcggccgagacctacaccaaggtcagcgagaccaccgcacacggatatccgatccgcgagcacatcaaccgcctcgtcgacggtgagatcatctgggcgcccgcgatcgacggtgcgttcgtgttgtcaacgcgcggcggtgatttcgacctgcagctcggcaccgacgtgtccatcggctacctgtcccatgacgccgaggtggtccacctctacatggaggagaccatgacgttcctgtgctacaccgctgaggcctctgtcgcgctgaccccc

SEQ ID NO: 2 - DNA, 24 bp, synthetic construct
  source 1..24: mol_type = other DNA
gattacaaggatgacgacgataag

SEQ ID NO: 3 - DNA, 804 bp, synthetic construct
  source 1..804: mol_type = other DNA
ttgaatcgaactaatattttttttggtgaatcgcattctgactggttgcctgtcagaggcggagaatccggtgattttgtttttcgacgtggtgacgggcatgccttcgcgaaaatcgcacctgcttcccgccgcggtgagctcgctggagagcgtgaccgcctcatttggctcaaaggtcgaggtgtggcttgccccgaggtgatcaactggcaggaggaacaggagggtgcatgcttggtgataacggcaattccgggagtaccggcggctgatctgtctggagcggatttgctcaaagcgtggccgtcaatggggcagcaacttggcgctgttcacagcctatcggttgatcaatgtccgtttgagcgcaggctgtcgcgaatgttcggacgcgccgttgatgtggtgtcccgcaatgccgtcaatcccgacttcttaccggacgaggacaagagtacgccgcagctcgatcttttggctcgtgtcgaacgagagctaccggtgcggctcgaccaagagcgcaccgatatggttgtttgccatggtgatccctgcatgccgaacttcatggtggaccctaaaactcttcaatgcacgggtctgatcgaccttgggcggctcggaacagcagatcgctatgccgatttggcactcatgattgctaacgccgaagagaactgggcagcgccagatgaagcagagcgcgccttcgctgtcctattcaatgtattggggatcgaagcccccgaccgcgaacgccttgccttctatctgcgattggaccctctgacttggggttga

SEQ ID NO: 4 - DNA, 806 bp, synthetic construct
  source 1..806: mol_type = other DNA
ccgaactgcatccgttccgcgagatcgacggtggacgtcaccacgcacccgcgactccgggcgatctgctgttccacctgcgtgccgaatccatggacgtgtgtttcgaactcgcgaccaagctcgtcgaggccatgtccggagccatcacgatcgtcgacgagacgcacggattccggttcttcgacaaccgtgacctcatggggttcgtcgacggtaccgagaacccggacggcaacctcgcggtcgtcgcgacccagatcggcgacgaggacccggatttcgctggcggctgctatgtgcacgtgcagaagtacctgcacgacatggcttcctggaactcgctgtccgtcgaggagcaggaacgcgtgatcggccgcaccaagctcgacgacatcgagttggacgacgacgtcaaacccgccaattcgcatgtggcgctcaacgtcatcgaggacgaggacggcaacgagctcaagataattcggcacaacatgccgttcggcgagatcggcaagggcgagttcggtacgtactacatcggttactcgcgcacacccagcgtcaccgaacgcatgctcgacaacatgttcatcggtgacccgccgggcaacaccgaccgcatcctggatttctccaccgccatcaccggcggactgttcttcacccccaccgtcgacttcctcgacgacccaccacctcttccgtcggaggacgaccgtgccgaaccggcttcggcgccttcggccgacccggtccacaccgacggctcactcggaatcggcagcctgaaaggaacccgctga

SEQ ID NO: 5 - DNA, 1578 bp, synthetic construct
  source 1..1578: mol_type = other DNA
tgaccccctgaccccgtagcgggtcaccccgcaccgcgagcgagtcaccccgcaccgcgagcgtgcgtgtctgctgcccggcacaccgcgaatcagcagcactcggcgcacgctcgtggatggcgagagtgcgcagagtgctcagctcggcggactcagaagcgaaatcagaccgacaggatggcgtcgagcgcggagtagaacaggcccagtccgtcgtcggacggtccggtcaacgcctcggtggcgtgttcgggatgcggcatgaggccgacgacgcggcgattctccgagcagatgcccgcgatgtcgcgcatcgacccgttgaggttctcgcgatagcggaagaccacgcggtcctcgccctcgagttcgtcgagcacggactcggacgccacgtagcgcccctcgccggacttcagcgggatcagcaggtcggcaccggtctcgtagcgcgtggtccacgccgaggtgttggacgccacttcgagccacacgtcgcggcagacgaagtgcagacccgcgttgcgggtcagcgcgccgggcagcaggccggcctcgcacagcacctggaagccgttgcagatgccgagcacgggcatgcccttgttggcggcctcgaccaccgagcccatcacgggcgcgaacttcgcgatggcgccgcaccgcaggtagtccccgtaggagaaaccgccgggcaccacgaccgcgtcgacgccgtgcagatccgcgtcggcgtgccacaggctcacggcctcagcacccgccagacggaccgcgcgggccgcgtcgatgtcgtccagcgtgccggggaacgtgatcacgcccacacgagtggtcatgcgtcctcccggctcacagtgaagtcctcgatgacggtgttcgccaggagcgattccgcgatctcgttgagggtttcgtcggtcacggaatcgtcgacctcaagctcaaaacgcttgccctgccgaacgtctgatatgcctttgtgcccgagtcggccaagcgctcccacgatggcctgaccttgcgggtcgaggatctcagccttgggcatgacgtgcacaaccacctttgccacggcgttcactctaccggcacgatcccaggtaggcgtcacacagtggtgcggccatcgcgtcgagcacctggctatcgtccaccgacggccacggcacctgcggcggggtcgtcgaccacagcgcgagctccacgacggtgctgttgttggggtcggcgagcaaatactggcgcagaacccggtcgccgaaggcgatcaccccggccagccggcccggttcgtcggtggtgagcgacggtgagctgtcgggcgccaccagctgacacgcccggatcgcggcggccgacgcgctcacggcctccatcgcgagctgcccgccccgccaggtctcgccgcgccagtggatgatctgcgcggagagctgccactgccccaccggccccgggccacctcggcccgcgcgctcaccgcatagtgccgcggatcgtcgggcagcagcggcagcgcgcactcctgttcgaaccggaaccgcggcgacaccgtcgtcacggccaaaccgg

SEQ ID NO: 6 - DNA, 45 bp, synthetic construct
  source 1..45: mol_type = other DNA
ttgcatgcctgcaggtcgacccgaactgcatccgttccgcgagat

SEQ ID NO: 7 - DNA, 64 bp, synthetic construct
  source 1..64: mol_type = other DNA
ttctcacttatcgtcgtcatccttgtaatcgctacccccgggggtcagcgcgacagaggcctca

SEQ ID NO: 8 - DNA, 49 bp, synthetic construct
  source 1..49: mol_type = other DNA
tggggttgaactagtgatcgatatctgaccccctgaccccgtagcgggt

SEQ ID NO: 9 - DNA, 40 bp, synthetic construct
  source 1..40: mol_type = other DNA
gtacccggggatcctctagaccggtttggccgtgacgacg

SEQ ID NO: 10 - DNA, 59 bp, synthetic construct
  source 1..59: mol_type = other DNA
attacaaggatgacgacgataagtgagaattctagaggtccgctttgaatcgaactaat

SEQ ID NO: 11 - DNA, 43 bp, synthetic construct
  source 1..43: mol_type = other DNA
gatatcgatcactagttcaaccccaagtcagagggtccaatcg

SEQ ID NO: 12 - DNA, 25 bp, synthetic construct
  source 1..25: mol_type = other DNA
tctagaggatccccgggtaccgagc

SEQ ID NO: 13 - DNA, 22 bp, synthetic construct
  source 1..22: mol_type = other DNA
gtcgacctgcaggcatgcaagc

SEQ ID NO: 14 - DNA, 26 bp, synthetic construct
  source 1..26: mol_type = other DNA
ccatttgtgcccgcaccacagcctca

SEQ ID NO: 15 - DNA, 34 bp, synthetic construct
  source 1..34: mol_type = other DNA
gccaaaaggcacgaatgtccgaaatgggtcagtt

SEQ ID NO: 16 - DNA, 20 bp, synthetic construct
  source 1..20: mol_type = other DNA
ctcacatgttctttcctgcg

SEQ ID NO: 17 - DNA, 20 bp, synthetic construct
  source 1..20: mol_type = other DNA
atcggcaagggcgagttcgg

SEQ ID NO: 18 - DNA, 20 bp, synthetic construct
  source 1..20: mol_type = other DNA
agcacatcaaccgcctcgtc

SEQ ID NO: 19 - DNA, 20 bp, synthetic construct
  source 1..20: mol_type = other DNA
gatcgaccttgggcggctcg

SEQ ID NO: 20 - DNA, 20 bp, synthetic construct
  source 1..20: mol_type = other DNA
gcaccgcaggtagtccccgt

SEQ ID NO: 21 - DNA, 20 bp, synthetic construct
  source 1..20: mol_type = other DNA
gaggtggtgggtcgtcgagg

SEQ ID NO: 22 - DNA, 20 bp, synthetic construct
  source 1..20: mol_type = other DNA
ccgccggtactcccggaatt

SEQ ID NO: 23 - DNA, 20 bp, synthetic construct
  source 1..20: mol_type = other DNA
cacgtcatgcccaaggctga